(19) United States
(12) Patent Application Publication
(10) Pub. No.: US 2015/0297365 A1
(43) Pub. Date: Oct. 22, 2015

(54) FLEXIBLE PROSTHETIC APPLIANCE

(71) Applicant: Worcester Polytechnic Institute, Worcester, MA (US)

(72) Inventors: Christopher A. Brown, Cazenovia, NY (US); John Foy, Worcester, MA (US); Alexander Verrelli, Worcester, MA (US); Sarah Basehore, Worcester, MA (US); Chadwick Whitcher, Worcester, MA (US); Jerrod Heiser, Worcester, MA (US); Tyler Bouchard, Worcester, MA (US)

(21) Appl. No.: 14/688,421

(22) Filed: Apr. 16, 2015

Related U.S. Application Data
(60) Provisional application No. 61/980,703, filed on Apr. 17, 2014.

(51) Int. Cl. A61F 2/66; A61F 2/60

(52) U.S. Cl. CPC A61F 2/6607; A61F 2/60; A61F 2002/5007

(57) ABSTRACT

A prosthetic appliance disposes a plurality of elongated, resilient members in a circular configuration around a pivot point representing a skeletal joint such as an ankle, wrist or shoulder. The resilient members are resilient and adapted to deformable respond to pivoting forces of the fulcrum, and forcibly deform in response to the pivoting of the shaft forcing the fulcrum against the resilient members. The circular orientation of the resilient members pivot the shaft orthogonal to the base from which the resilient members extend, such that the resilient members bias the fulcrum and shaft in the orthogonal position at a rest position. Forces drawing the shaft off-center disposed the fulcrum against one or more of the resilient members and causing resilient deformation and counterforces against the fulcrum back to the centered rest position. In response to pivoting movement, the resilient members apply increasing forces for biasing the pivoting shaft.

### RELATED APPLICATIONS

This patent application claims the benefit under 35 U.S.C. §119(e) of U.S. Provisional Patent App. No. 61/980,703, filed Apr. 17, 2014, entitled “FLEXIBLE PROSTHETIC APPLIANCE,” incorporated by reference in entirety.

### BACKGROUND

Major limb amputations have been increasing in recent years. This is due, at least in part, to an aging baby boomer population and increased longevity from advanced health care availability. Many amputations result from diabetes, which becomes more prevalent in older population ranges. Peripheral arterial disease and traumatic events are also major contributors. Many such amputations affect the lower leg below the knee region.

### SUMMARY

A prosthetic appliance disposes a plurality of elongated, resilient members in a circular configuration around a shaft attached to a pivot point representing a skeletal joint such as an ankle, wrist or shoulder. The resilient members are adapted to deformably respond to pivoting forces of a fulcrum at the end of the shaft, and forcibly deform in response to the pivoting of the shaft that dispose the fulcrum against the resilient members. The circular orientation of the resilient members pivot the shaft generally orthogonal to the base from which the resilient members extend, such that the resilient members bias the fulcrum and shaft in the orthogonal position at a rest position. Forces drawing the shaft off-center dispose the fulcrum against one or more of the resilient members and cause resilient deformation and counterforces against the fulcrum back to the centered rest position. In this manner, an at rest prosthetic emulating a ankle is disposed substantially upright, and in response to pivoting off vertical, the resilient members apply increasing forces for biasing the pivoting shaft defining the prosthetic ankle back towards a vertical rest position.

Conventional approaches to prosthetic devices available for below knee amputees are designed to imitate a normal human foot and ankle. Common designs involve carbon fiber leaf springs that serve as a foot. The problem with a simple carbon fiber leaf spring rigidly attached to prosthetic tibia (shinbone) structure is that it only recovers around 90% of the transferred energy that can be translated into forward momentum. Higher performance models include hydraulic systems with a motor or energy source such as a lithium ion battery. These devices can be heavy and cause discomfort to the user's residual limb. They also are very expensive and fall outside of medical insurance packages, putting increased burden on the patient. The passive response provided by the resilient members avoids the expense and complexity of active systems such as hydraulic, pneumatic or servo motor approaches, and also avoids an exaggerated, unnatural response that active systems tend to provide.

Configurations herein employ prosthetic appliance for an ankle replacement having pivotally attached opposed polygonal plates having elongated motion limiting members (resilient members) coupling corresponding sides of the opposed polygonal plates, in which the coupling has a fixed, pivotal attachment at one of the polygonal plates and slideable communication at the opposed polygonal plate defining the fulcrum. The motion limiting members are adapted to resiliently deform in response to pivotal movement between the plates, such as when an ankle flexing disposes the pivot in a direction, causing resistance by the motion limiting member in the disposed direction. The motion limiting members may substantially equal resiliency for biasing the opposed polygonal plates in a parallel orientation at a rest position, typically an upright position corresponding to a standing subject or patient. At least one of the polygonal plates is adapted for prosthetic connection to a subject limb such as a tibia below the knee position, in which the rest position is defined by an immobile state of the subject limb.

Configurations disclosed herein address individual functions and needs of the prosthetic foot, toe, and ankle. In the example arrangement, the prosthetic ankle includes a ball joint, and cantilever springs with pre-stressed components. The ball joint allows the user to rotate the prosthetic in all directions and to apply movement in each direction. The shaft attached to the ball joint connects with an octagonal (polygonal) plate, or member, and each side has a separate cantilever spring defining the motion limiting member. The resilient members may be cut at a gradient thickness that is thick at the bottom (nearest the foot) and thinner at the top (knee side), near the contact point with the rod of the ball joint. This allows for gradually increased resistance as the user bends the ankle. Also, the springs will be pre-stressed so that there will need to be a minimum force requirement to being movement.

In further detail, the motion limiting members are elongated, resilient cantilevered springs biased in a direction supporting upward orientation of a shaft connecting the polygonal plates, in which the shaft is disposed in a direction based on a supported skeletal member (upwards, in the case of a tibia, or ankle). The cantilevered members have a tapered thickness for varying a resistive force in response to increased pivotal movement off center from a rest position, such that the rest position disposes the cantilevered members substantially upright around the centered polygonal plate.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 shows a context view of the prosthetic appliance structure as defined herein;

FIG. 2 shows a deployed view of the appliance of FIG. 1;

FIG. 3 shows an isometric view of a configuration of the appliance in FIG. 1 having 8 resilient members;

FIG. 4 shows a top view of the appliance of FIG. 3;

FIG. 5 shows a side view of the appliance of FIG. 3; and

FIG. 6 shows a perspective view of the appliance of FIG. 3.

### DETAILED DESCRIPTION

Configurations discussed below depict a pivotal joint biased around a central orientation by resilient members functioning as cantilever beams against a fulcrum disposed on a pivoting shaft defining the pivotal joint for engagement when the pivoting shaft experiences forces that dispose it off-center away from an orthogonal orientation with a base from which the cantilever beams extend. Any number of cantilever beams may be employed around a pivotal member, as will be discussed further below. An example depicts a prosthetic ankle configuration, however the disclosed structure is applicable to other applications of a passive self-centering pivotal shaft is beneficial.

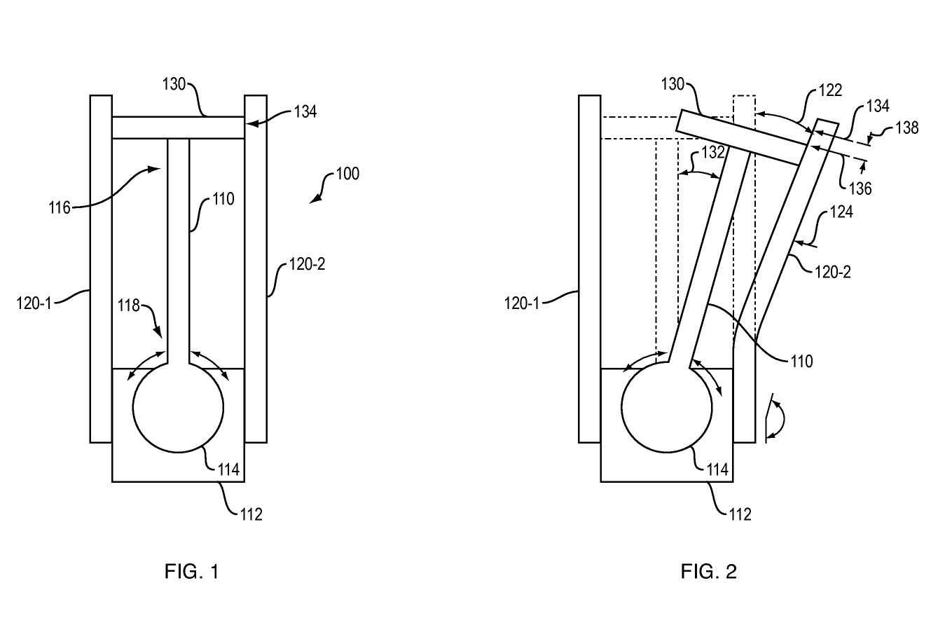

FIG. 1 shows a context view of the prosthetic appliance structure as defined herein. Referring to FIG. 1, in the structure 100 defined by the prosthetic appliance, a shaft 110 pivotally extends from a base 112 at a pivotal attachment such as a ball joint 114. A plurality of elongated, resilient members 120-1 . . . 120-2 (120 generally) extend from the base 112 parallel to the shaft 110. The shaft 110 is attached to a fulcrum 130 at an opposed, distal end 116 of the shaft 110 from the ball joint 114. The fulcrum 130 is flanked by the resilient members 120 for maintaining the shaft 110 substantially orthogonal to the base 112.

A particular deployment involves a prosthetic ankle appliance, having a shaft 110 pivotally attached to a base 112, such that the shaft 110 is adapted to pivot a prosthetic receptacle around the base 112. The shaft 110 attaches to a receptacle adapted to receive a host appendage, typically a surgically amputated leg. The base 112 is further enhanced by a foot emulating structure (discussed below). The base 112 has a plurality of elongated resilient members 120 (2 shown in FIGS. 1 and 2) secured to the base 112 and extending substantially parallel to the shaft 110, and a fulcrum 130 attached to the shaft 110 and configured for engaging at least one of the resilient members 120 in response to pivoting of the shaft 110, such that the resilient members 120 are deformable in response to the engagement for biasing the shaft to parallel orientation with the resilient members 120 by exerting a counterforce based on the elasticity of the resilient members 120.

FIG. 2 shows a deployed view of the appliance of FIG. 1. Upon pivoting of the shaft 110, the fulcrum 130 is disposed against at least one of the resilient members 120-2, and deflects the resilient member 120-2 according to a deflection angle 132. The resilient member 120 responds with a deformation angle 122, while the resilient member 120-2 operates as a cantilever beam for applying force 124 against the pivot to return the shaft 110 orthogonal to the base (upright, as shown in FIGS. 1 and 2). The fulcrum 130 engages the resilient member 120 in a slideable manner, and as the resilient member 120-2 increases the angle of deflection 122, contact from the fulcrum 130 at a fulcrum point 134 slides down the resilient member, from an initial point 134 to a deployed position at fulcrum point 136, reducing the effective length of the resilient member by a distance 138 and thus increasing the centering force 124 due to a shorter cantilever beam. Therefore, during pivoting, the fulcrum 130 slideably engages at least one resilient member 120 in response to the pivoting, such that the slideable engagement decreases a distance 138 of the fulcrum 130 from the point of engagement 134 to a point of attachment to the base 112. Due to the effective decreasing length of the cantilever, the resilient members 120 exert an increasing biasing force against the shaft in response to an increased pivot angle 122 relative to the base 112.

A particular configuration includes a prosthetic ankle appliance, now discussed below, however alternate configurations may address other skeletal joints or non-rehabilitative uses for biasing a 360 degree pivotal member in a substantially upright position through the use of circularly arranged cantilever beams.

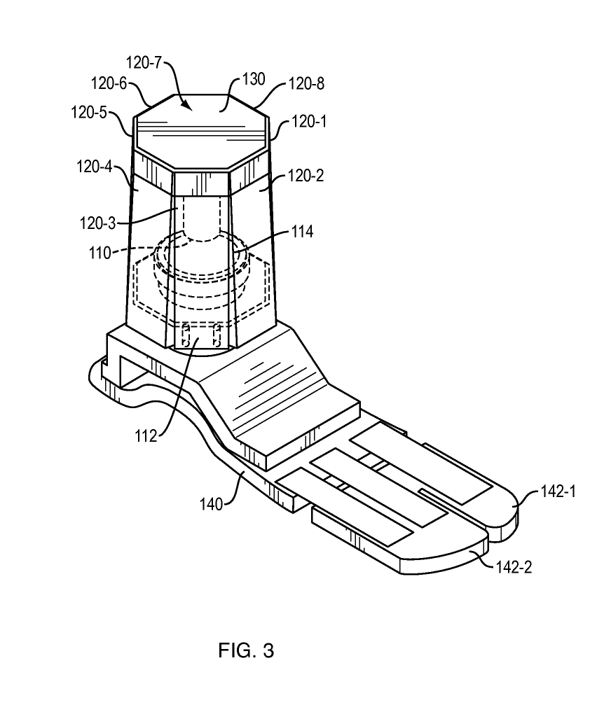

FIG. 3 shows an isometric view of a configuration of the appliance in FIG. 1 having eight (8) resilient members. In the example of FIG. 3, the fulcrum 130 is defined by an octagonal plate centered around the shaft 110, and is adapted to engage a plurality of the resilient members 120 based on a direction of the pivot. The resilient members 120-1 . . . 120-8 are disposed equidistantly around the shaft 110 for engaging the fulcrum 130 in proportion to a component of the pivot directed toward each resilient members 120 of the plurality of resilient members 120. The base 112 is attached to a foot member to emulate a walking function to a patient or wearer of the appliance. The circular arrangement provides a response in 360 degrees of pivoting range, however a pivot in any direction will trigger a response by multiple adjacent resilient members depending on the direction of the force. For example, in FIG. 3, a forward pivot (toward toes 142 of the foot 140) will most significantly affect resilient member 120-2, but adjacent resilient members 120-1 and 120-3 will also receive a proportion of the pivot force proportional to the facing of the side of the base 110.

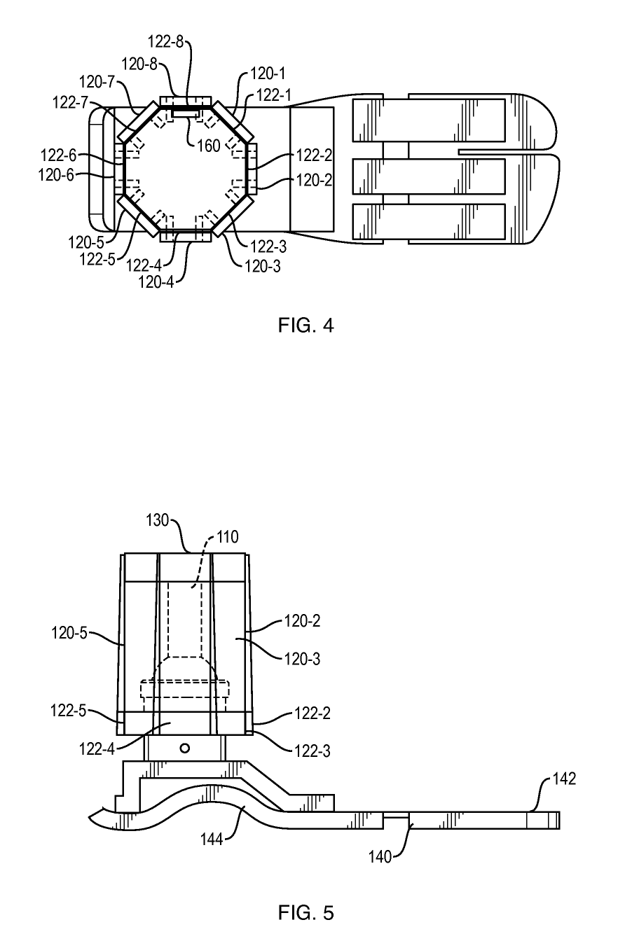

FIG. 4 shows a top view of the appliance of FIG. 3. Referring to FIGS. 3 and 4, the example of FIG. 3 employs an octagon for covering every 45 degrees of a circle, however any number of sides may be employed. The fulcrum 130 may be defined by a polygon extending around, and substantially centered on, the shaft 110, such that the polygon has a plurality of sides 122-1 . . . 122-8, each side corresponding to a resilient member 120-1 . . . 120-8. The example fulcrum 130 is therefore is a polygon shape, such that each side 122 of the polygon provides a fulcrum point disposed toward the corresponding resilient member 120.

The base 112 and fulcrum 130 arrangement therefore defines pivotally attached opposed polygonal plates having elongated motion limiting members, or resilient members, coupling corresponding sides 122 of the opposed polygonal plates, in which the coupling has a fixed attachment at one of the polygonal plates (base 112) and slideable communication at the opposed polygonal plate (fulcrum 130), in which the motion limiting members are adapted to resiliently deform in response to pivotal movement between the plates. In a particular configuration, the motion limiting members having substantially equal resiliency for biasing the opposed polygonal plates in a parallel orientation at a rest position, and at least one of the polygonal plates attaches to a receptacle adapted for prosthetic connection to a subject limb, in which the rest position is defined by an immobile state of the subject limb, typically resting upward. Alternatively, the resilient members may be configured for a varied resistance depending on the direction of pivot, such as by varying the thickness, discussed further below.

In the example shown, the resilient members are elongated, cantilevered beams attached to the base, however the resilient members may also be tension, compression or leaf springs in addition to cantilever beams. The disclosed configuration depicts a passive system, in which resistive forces emanate responsively only from the compression or tension of the beams, however active resiliency may also be employed. For example, the resilient members may be elastic, hydraulic or electromagnetic, and may be responsive to external control such as from a processor or robotic driven system, and may be adapted for conditioned, learned response.

FIG. 4 illustrates an additional feature wherein each side 120 of the polygon (fulcrum) 130 has a receptacle 160 adapted to slideably engage the resilient member 120 for maintaining pivotal movement orthogonally toward the direction of deformation of the resilient member 120. The receptacle 160 engages a protraction on the fulcrum 130 for aligning the fulcrum 130 against a surface on the resilient member 120 for ensuring that direct perpendicular forces are applied and avoiding side forces tending to “twist” or laterally dispose the resilient member 120.

Depending on the response desired, the cantilevered (resilient) members 120 may employ a tapered thickness for varying a resistive force in response to increased pivotal movement off center from a rest position, in which the rest position disposes the cantilevered members 120 substantially upright around the centered polygonal plate, or fulcrum 130. For example, since a walking movement tends to generate greater forces in the direction of travel, resilient members facing the toe and heel might be thicker for providing a greater resistive force 124

FIG. 5 shows a side view of the appliance of FIG. 3. Referring to FIGS. 4 and 5, the base 110 attachment to the foot 140 is shown. The foot 140 may include a leaf spring 144 for absorbing or dampening heel-to-toe forces as an active prosthetic appliance is employed for walking.

The foot 140 defines a ground interface, and one or more resilient extensions 142 define prosthetic toes. The resilient extension 142 is responsive to foot 140 movement away from flush engagement with a planer surface (as in a walking motion), in which the resilient extension is adapted to flexibly deform for biasing the foot away from flush engagement with the planar surface in response to the perpendicular force from the polygonal plate. For example, during a walking motion, the resistance to or stiffness of forward and backward flexing (dorsi and plantar flexion) of the ankle should be different as these should be different from roll, and that inward and outward roll might differ, calling for different resilience or “stiffness” in the resilient members (i.e. thicker beams) to appropriately counter the walking forces. The ground interface is defined by a prosthetic foot for simulating a walking response whereby the ground interface emulates the foot and arch and the resilient extensions 142 define the toes.

The toe design is independent from the foot, in contrast to conventional designs that have the toe and foot in a combined material. The advantage allows design material that is better suited for the needs of the toes, to allow different flexing characteristics than from the foot as a whole.

The foot 142 design includes a leaf spring of carbon fiber or other suitable material in conjunction with rounded heel that will allow for a rolling motion during walking that lessens energy loss upon impact and also provides for reduced impact force on the residual limb. Alternate materials may also be employed, and the rounded heel is operable in conjunction with the pivoting ankle to moderate forces. In the example shown, the foot prosthetic is provided by a base attached to one of the polygonal plates, and substantially perpendicular to the shaft at a rest position. The leaf spring 144 defines the coupling between the base and the polygonal plate, such that the leaf spring 144 is adapted to deform in response to a substantially perpendicular force against the base from the polygonal plate.

The base 112 and foot 144 structure further comprises one or more resilient extensions 142-1 . . . 142-2 for defining the toes, such that the resilient extensions 142 are responsive to foot movement away from flush engagement with a planer surface (i.e. floor). The resilient extension is adapted to flexibly deform for biasing the base towards flush engagement with the planar surface in response to the perpendicular force from the polygonal plate, providing a bias or springlike resistance to angled movements of the toes.

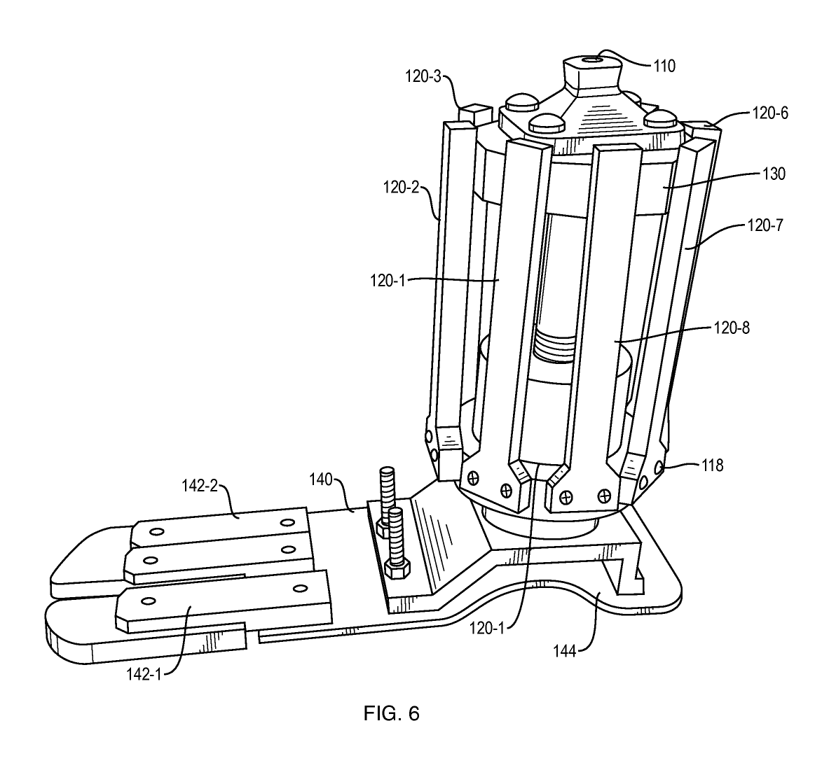

FIG. 6 shows a perspective view of the appliance of FIG. 3. Referring to FIGS. 1, 2 and 6, at a rest position, the shaft 110 representing the ankle position is disposed upwards, centered between the non-deformed resilient members 120. The undeformed resilient members 120 define a rest position of the shaft 110 that orients the shaft substantially orthogonal to the base 112, due to the biasing forces directing the shaft to the orthogonal orientation.

In the example configuration, the resilient members 120 attach to the base 112 by a plurality of screws. Any suitable attachment may be performed for providing a rigid connection to withstand the cantilever force experienced by the deformed beam maintains the resilient centering force. The resilient members 120 define a cantilever spring rigidly attached to the base at a proximate 118 end and adapted to deform or flex along its length in response to a force applied at an opposed end 116, where the magnitude of force require to displace or deform the beam is proportional to the distance from the attachment at which the force is applied.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. A prosthetic ankle appliance, comprising:
a shaft attached to a base so that it can pivot or tilt relative to the base about one or more axes, the shaft adapted to pivot around the base;
a plurality of resilient members secured to the base and extending substantially parallel to the shaft; and
a fulcrum attached to the shaft and configured for engaging at least one of the resilient members in response to pivoting or tilting of the shaft, the resilient members deformable in response to the engagement for biasing the shaft to parallel orientation with the resilient members.

2. The appliance of claim 1 wherein the shaft has an interface for a prosthetic receptacle at the fulcrum end of the shaft opposed from the base, the receptacle adapted for engaging an appendage of a user for prosthetic enhancement.

3. The appliance of claim 2 wherein the fulcrum extends around the shaft and is adapted to engage a plurality of the resilient members based on a direction of the pivot or tilt.

4. The appliance of claim 3 wherein resilient members are disposed equidistantly around the shaft for engaging the fulcrum in proportion to a component of the pivot directed toward each resilient members of the plurality of resilient members.

5. The appliance of claim 4 wherein the fulcrum is defined by a polygon extending around, and substantially centered on, the shaft, the polygon having a plurality of sides, each side corresponding to a resilient member.

6. The appliance of claim 5 wherein the fulcrum is a polygon shape, each side of the polygon providing a fulcrum disposed toward the corresponding resilient member.

7. The appliance of claim 6 wherein each side of the polygon has a receptacle adapted to slideably engage the resilient member for maintaining pivotal movement orthogonally toward the direction of deformation of the resilient member.

8. The appliance of claim 1 wherein the fulcrum slideably engages at least one resilient member in response to the pivoting, the slideable engagement decreasing a distance of the fulcrum from the point of engagement to a point of attachment to the base.

9. The appliance of claim 8 wherein the resilient members exert an increasing biasing force against the shaft in response to an increased pivot angle relative to the base.

10. The appliance of claim 1 wherein the undeformed resilient members define a rest position of the shaft that orients the shaft substantially orthogonal to the base, the biasing forces directing the shaft to the orthogonal orientation.

11. The appliance of claim 10 wherein the resilient members define a cantilever spring rigidly attached to the base at a proximate end and adapted to deform or flex along its length in response to a force applied at an opposed end, where the magnitude of force require to displace or deform the beam is proportional to the distance from the attachment at which the force is applied.

12. The appliance of claim 11 wherein the resilient members are elongated cantilevered beams adapted to forcibly deform in response to pivoting of the shaft.

13. The appliance of claim 10 wherein the resilient members provide a passive, self centering response to pivotal forces for biasing the shaft orthogonal to the base, the passive response driven only by compressive and tensile forces generated by the resilient members in response to displacing forces of the shaft and fulcrum.

14. A method of providing passive self-centering prosthetic ankle appliance, comprising:
disposing plurality of resilient members arranged in a circularly opposed arrangement around a fulcrum attached to a pivoting shaft extending from a base,
the resilient members defining a cantilever spring rigidly attached to the base at a proximate end and adapted to deform or flex along its length in response to a force applied at an opposed end, where the magnitude of force require to displace or deform the beam is proportional to the distance from the attachment at which the force is applied.

15. A prosthetic appliance comprising:
pivotally attached opposed polygonal plates having elongated motion limiting members coupling corresponding sides of the opposed polygonal plates, the coupling having a fixed attachment at one of the polygonal plates and slideable communication at the opposed polygonal plate, the motion limiting members adapted to resiliently deform in response to pivotal movement between the plates;
the motion limiting members having substantially equal resiliency for biasing the opposed polygonal plates in a parallel orientation at a rest position, and
at least one of the polygonal plates adapted for prosthetic connection to a subject limb, the rest position defined by a resting state of the subject limb.

16. The appliance of claim 15 wherein the motion limiting members are resilient cantilevered springs biased in a direction supporting upward orientation of a shaft connecting the polygonal plates, the shaft disposed in a direction based on a supported skeletal member.

17. The appliance of claim 16 wherein the polygonal plates are octagonal plates.

18. The appliance of claim 15 wherein the pivotal attachment further comprises a ball joint coupling to one of the polygonal plates and a shaft extending from the ball joint to the opposed polygonal plate, the ball joint adapted to provide pivotal movement of the opposed polygonal plate around the polygonal plate at a distance defined by the length of the shaft.

19. The appliance of claim 18 further comprising a ground interface attached to one of the polygonal plates, and substantially perpendicular to the shaft at a rest position.

20. The appliance of claim 19 further comprising a leaf spring coupling between the ground interface and the polygonal plate, the leaf spring adapted to deform in response to a substantially perpendicular force against the ground interface from the polygonal plate.

21. The appliance of claim 20 wherein the ground interface further comprises a resilient extension, the resilient extension responsive to movement away from flush engagement with a planer surface, the resilient extension adapted to flexibly deform for biasing the ground interface periodically away from flush engagement with the planar surface in response to the perpendicular force from the polygonal plate, the ground interface defined by a prosthetic foot.

22. The appliance of claim 16 wherein the cantilevered springs have a tapered thickness for varying a resistive force in response to increased pivotal movement off center from a rest position, the rest position disposing the cantilevered members substantially upright around the centered polygonal plate.

23. The appliance of claim 15 wherein a first motion limiting member is adapted to apply a different force than a second motion limiting member for varying a centering force applied based on the direction of the pivot.

24. The appliance of claim 2 further comprising a rotational linkage in the interface for allowing independent rotation of the receptacle.
